(19) United States
(12) Patent Application Publication
(10) Pub. No.: US 2005/0164407 A1
(43) Pub. Date: Jul. 28, 2005

(54) Increasing the sensitivity and specificity of nucleic acid chip hybridization tests

(76) Inventors: Cord F Stahler, Weinheim (DE); Peer F. Stahler, Mannheim (DE); Markus Beier, Heidelberg (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/505,898

(22) PCT Filed: Feb. 26, 2003
(86) PCT No.: PCT/EP03/01972

(30) Foreign Application Priority Data
Feb. 28, 2002 (DE) 102087709

(51) Int. Cl. G01N 33/543

(52) U.S. Cl. 436/518

(57) ABSTRACT

The invention relates to a method of increasing the sensitivity and specificity of nucleic acid chip hybridization tests and to devices suitable for carrying out the inventive method.

The invention relates to a method of increasing the sensitivity and specificity of nucleic acid chip hybridization tests and to apparatuses suitable for carrying out said method.

DNA hybridization is based on the sequence-specific formation of a complementary double strand from different single-strand sources under particular experimental conditions. If a single strand sequence is known and utilized as a probe (for example in the form of an oligonucleotide), it is possible, after detection of a hybridization event, for example via labeling with a dye, to derive the target sequence. This process is reversible and may be controlled via changes in temperature. This property of DNA is utilized in various applications, for example in DNA sequence decoding (Sequencing by hybridization, SbH) or in measuring the activity of different cells or of different cellular states (Expression Profiling or Gene Expression Monitoring), by determining the copy number of DNA transcripts (mRNA) which are present in a cell at a defined time. In this connection, hybridization events of single-stranded DNA and of mRNA need to be evaluated quantitatively. Another important application, a special case of sequencing, is the mutational analysis of individual DNA single positions (Single Nuclear Polymorphisms, SNP) which serve to elucidate diseases at a molecular level.

For practical applications, the so-called “nucleic acid chip technique” has been established as a promising tool for the abovementioned problems. This involves immobilizing hybridization probes of a known sequence, for example oligonucleotides or cDNA molecules, at defined locations on surfaces of a support, which probes are used there as capture molecules for target sequences from different sample material. Via detection of the hybridization of target sequences to said surfaces, for example by labeling the sample material with fluorescent dyes, a hybridization experiment produces measured signals which may be evaluated with the aid of suitable methods. Owing to the fact that the probe sequence is known, it is possible to identify and characterize the target sequences in the sample material.

Hybridization to a solid phase, for example a DNA chip, DNA array or DNA filter, is a diffusion-dependent process which depends on a complex combined action of various factors, inter alia
a) the reaction temperature,
b) the buffer conditions,
c) the relative single-strand concentrations,
d) the path length between the sample molecule and the probe and
d) the number of DNA-DNA interactions taking place on said path.

With practical application, for example in expression profiling, SbH and others, owing to the complexity of the DNA molecules in the sample material, to the usually large sample volumes and chip areas, strong limitations are encountered with respect to:
a) accuracy and/or specificity (quality),
b) sensitivity (amount of sample, complexity of sample material)
c) throughput (speed, costs) and
d) possible embodiments (utilization for research and diagnostics).

Nucleic acid hybridization is an equilibrium process which may be described by the law of mass action: [A] (probe)+[B] (target sequence)⇄[AB]. Since [A], i.e. the concentration of the probe immobilized on a chip, is, according to the prior art, usually approximately constant for all immobilized probes (A1 to An), problems arise for the relative quantification of target sequences (B) in sample mixtures (B1 to Bn), if [B1] to [Bn] (i.e. the concentration of the individual target sequences B1 to Bn) is not constant. This is the case, for example, in gene expression profiling. Individual target sequence concentrations may vary by a factor of 10 000. As a result of this, some probe locations on the chip may be physically saturated in an experiment, in comparison with other probe locations, and the linear dynamic measuring range is exceeded during detection, thereby rendering impossible a quantification of all signals in a single measurement. This has an influence on the sensitivity (quality) and possible applications (utilization).

In the SbH application, for example, there is the problem that, in the case of eukaryotic target sequences, a large amount of repetitive sequences (97% in the human genome) is present, as a result of which the relative concentration of the relevant target sequence region is almost by a factor of 100 lower than it could be, if, for example, the repetitive sequences were to be “filtered out” prior to the experiment. This has an influence on the sensitivity and specificity (quality) and impedes many conceivable applications using DNA chips.

Another problem of the SbH application is the fact that it is not possible in principle to specifically form any desired DNA double strands at a defined hybridization temperature (quality, utilization), since DNA hybridization is kinetically controlled and double strands form which do not correspond to the thermodynamic minimum. Only by reversibly dissolving unspecifically bound DNA molecules and by setting the individual duplex melting temperature, may the reaction in the direction of the thermodynamically most favorable state be made possible (specific double strand formation).

Studies on the determination of analytes on solid phases are known, as described, for example, by R. P. Ekins, in U.S. Pat. No. 5,432,099. The known DNA chip hybridization methods may be divided into two categories: the passive hybridization method and the actively supported hybridization method.

In passive hybridization, the sample solution is stationary and the hybridization is carried out at a defined temperature in a diffusion-dependent manner. This category includes the two-dimensional slide or array technique using chips which are prepared by spotting or in situ synthesis. These techniques have the advantage of having a relatively high location density. Disadvantageously, however, the use of large sample volumes is required, only a low local target sequence concentration causing, inter alia, a very slow hybridization (approx. 16-48 h) is produced and the usable linear measuring range covers only 2-3 orders of magnitude. Another disadvantage arises due to the two-dimensional uniform temperature which may result in unspecific (false-positive) hybridization results. In the case of SbH with repetitive DNA, the signal-to-background ratio ranges from low to not measurable in this technique.

In the case of the actively supported hybridization methods, the sample solution is moved through channels or, with the aid of electric fields, across the immobilized probes, and a temperature gradient can be set. This category includes the 3D chip technique with channel geometry. This technique is advantageous in that the hybridization times are short, due to the active movement of the sample, and that relatively small sample volumes can be utilized. Disadvantageously, however, the location density is low and a local temperature control cannot be set, which may result in false-positive events.

Another actively supported hybridization method is the “96-well printing” technique in microtiter plates. This technique has the advantage of the individual microtiter plate wells being individually thermally controllable, for example with the aid of a PCR apparatus. However, disadvantages are the use of very large sample volumes, the low location density and the diffusion-dependent and slow hybridization which strongly affects the sensitivity of this method.

Finally, the electronically controlled hybridization is known. This type of actively supported hybridization involves moving the sample toward the probe. As a result, the hybridization times obtained are very short. Due to the electronics, a temperature-equivalent stringency is produced, making a discrimination of false-positives possible. Nevertheless, this technique is quite expensive, has very low integration densities and cannot readily be used for SbH and genomic expression profiling.

In summary, it can be pointed out that previously established methods of detecting nucleic acids by hybridization using chips with immobilized hybridization probes cannot be adequately adapted to the complexity of biological sample material. It was therefore an object of the present invention to provide methods and systems for the determination of analytes on supports, for example chips, which methods and systems at least partially avoid the disadvantages of the prior art.

This object is achieved by a method in which individual regions or groups of regions of hybridization probes on the support are designed in a variable manner for the application desired in each case, thus considerably improving the sensitivity, specificity and economy. The method of the invention, however, enables not only the analytes to bind to a probe by hybridization but also other receptor-analyte bioaffinity interactions such as, for example, nucleic acid-protein, protein-protein, low molecular weight compound-protein or receptor-ligand bonds to be detected.

Thus, the object of the present invention is a method of determining analytes, comprising the steps
(a) providing a support having a plurality of predetermined regions at which in each case different receptors are immobilized on said support,
(b) contacting said support with an analyte-containing sample and
(c) determining the analytes via their binding to the receptors immobilized on the support,
   characterized in that, for predetermined regions or groups of regions with receptors in each case different conditions (i) for local receptor concentration, (ii) for receptor-ligand affinity, (iii) for kinetics of receptor-analyte interaction or/and (iv) for virtual analyte concentration are provided.

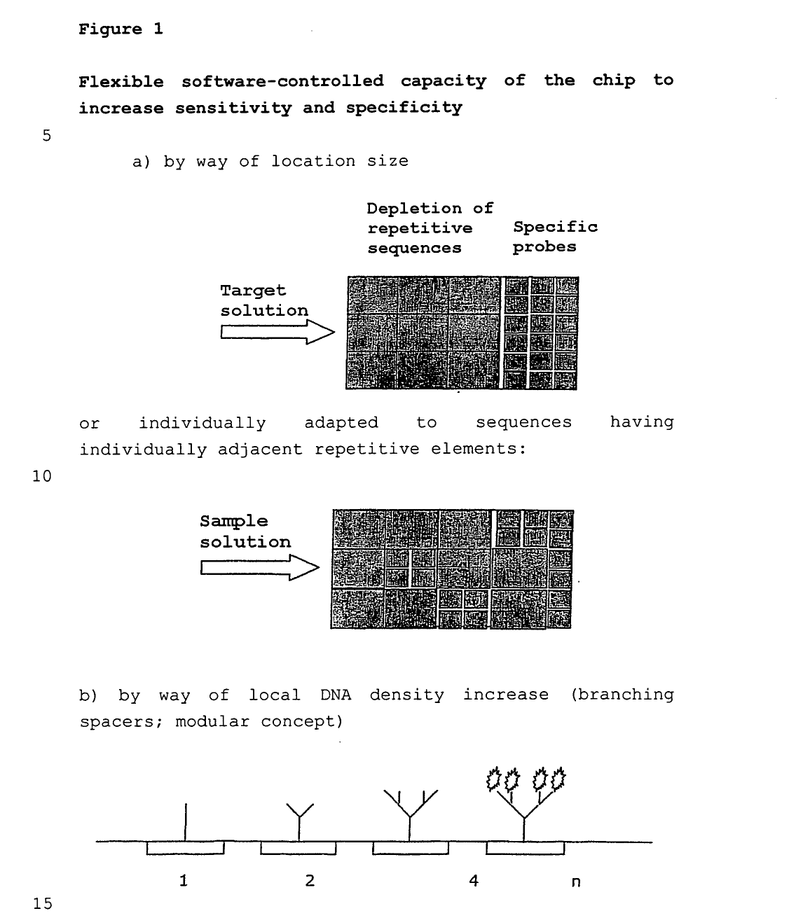

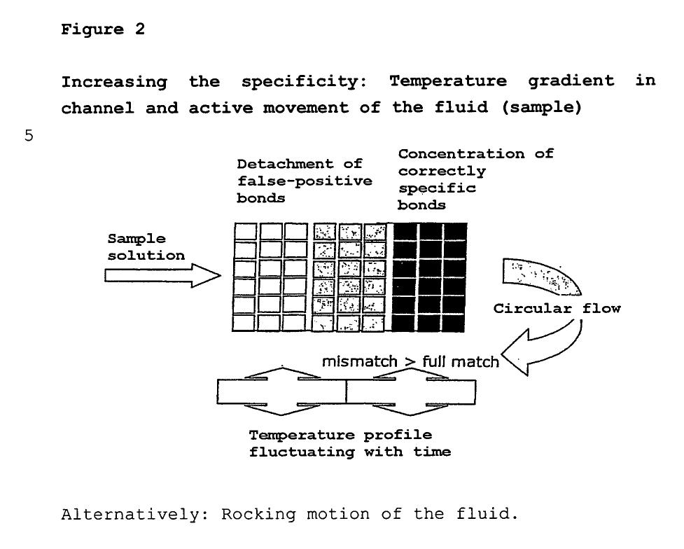

Preferably, the method of the invention is based on the Geniom® technology which is described in WO 00/13018. It may utilize the geometric structures (micro-channels), the flexible loading capacity of the fluid processor (i.e. the different local receptor concentrations as depicted in FIG. 1) and the possibility of active fluid movement in combination with the possibility of local temperature control (as depicted in FIG. 2). The different aspects may be employed individually or in combination, depending on the application.

Especially the production times of new DNA chips, which are particularly short with the Geniom® technology (within a few hours), and the short learning cycles made possible thereby render these applications with DNA chips not only technically feasible but also economical, such as, for example, sequencing (SbH) of DNA with a high proportion of repetitive DNA, expression profiling with a sufficient dynamic sensitivity range, in order to be able to record quantitatively both very rare and very common transcripts in complex mRNA samples, and massively parallel SNP detection with high individual oligo duplex specificity.

It is possible in the method of the invention, preferably by using the Geniom® technology, for example using an integrated synthesis and analysis system (ISA system), to vary biophysical parameters such as temperature and local and virtual concentration—alone or in combination—both in the preparation of a test and during a test (online detection) or/and in the evaluation of a test (learning system). This manipulation of the parameters influencing the hybridization signal may be carried out both globally and locally (i.e. individually for each oligonucleotide sensor).

Anmother object of the invention is an apparatus for determining an analyte, comprising a support having a plurality of predetermined regions at which in each case different receptors are immobilized on said support, characterized in that said predetermined regions with receptors have, at least partially, a different local receptor concentration. The apparatus of the invention is furthermore characterized in that means are provided in order to vary the kinetics of the receptor-analyte interaction or/and to vary the virtual analyte concentration in the predetermined regions.

The method of the invention may achieve an improvement, for example in the expression profiling application. Constitutively highly expressed gene sequences may be depleted over areas which are up to 100× larger than the others and have up to 10× higher location densities. This increases the sensitivity for rarely expressed genes. This sensitivity may be optimized by learning cycles, with a new chip being programmed for the genes identified in a first experiment, on the basis of relative frequency (fluorescence intensity), which chip evens out differences via the size or/and receptor density of the locations so as to produce a preferably homogeneous measured signal. The sensitivity may also be optimized by means of different times (amounts) of illumination per location, using a light source matrix. This illumination setting may then be used for studying test material. The system becomes more sensitive and the dynamic range is shifted into the linear range.

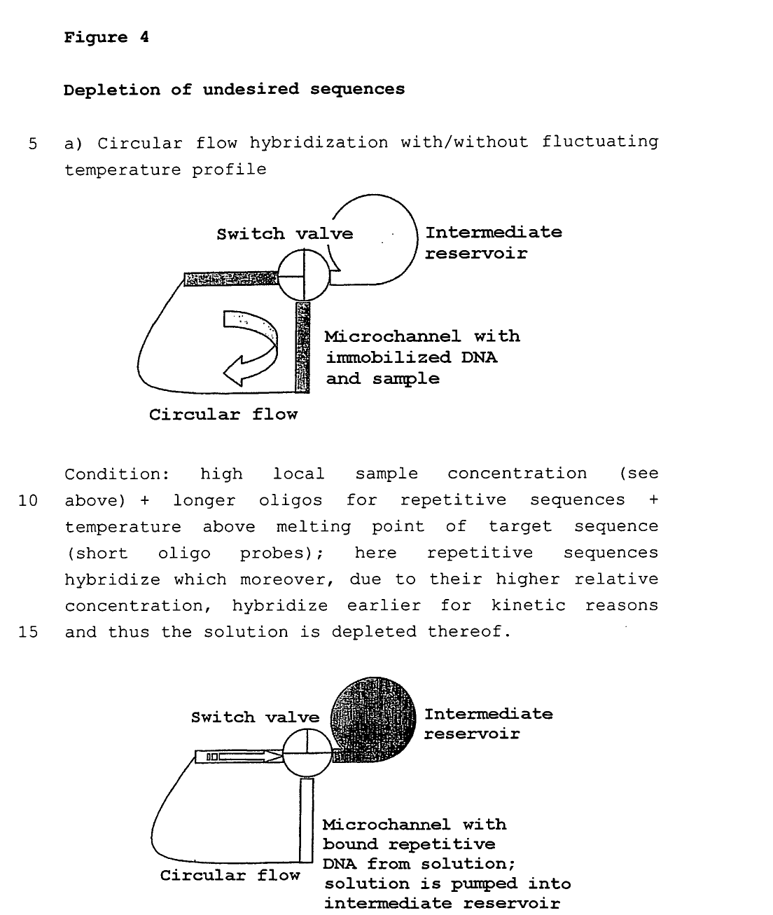

An improvement may also be achieved in the SbH application. Repetitive DNA may be depleted in regions of the chip by immobilizing, on suitably large surfaces, special spacers which have relatively high 3-dimensional branching and a relatively high local location density and thus “filter out” the repetitive sequences. This renders the actual measurement more sensitive and delivers a better signal-to-background ratio. This effect may be accelerated by very fast reassociation kinetics: a hybridization is carried out within one minute so that frequently occurring sequences can quickly find their probe. The solution is subsequently removed and stored intermediately in a reservoir (see FIG. 4). The hybridized DNA is removed with hot solution and detached. This cycle is repeated, until, after a plurality of such cycles, the hybridization solution can be introduced into the measuring channel or, possibly, into the same channel. Depending on the experiment, it is possible to specifically deplete specific repetitive sequences with the aid of the variable location density, the active movement of fluid and different local thermal control.

When a plurality of overlapping or nonoverlapping receptors for a gene are arranged in proximity of one another, it is possible, via differently sized surfaces of individual receptors, to utilize the effect of mass action in order to balance differences in affinity, i.e. the local concentration of an oligonucleotide probe is varied, meaning that the differences in the melting temperature of various oligonucleotides are compensated for, for example by individually adapting the location size. Thus, a correspondingly larger location area is assigned to an oligonucleotide probe with lower melting temperature, caused, for example, by a high AT content, than to a probe with a higher melting point, caused for example, by a higher GC content. During a subsequent signal quantification, the larger location areas may be integrated and assessed like a standard signal (learning principle).

Different melting points of receptor probes may also be adjusted by varying the area density, in addition to altering the area. This is accomplished, for example, by setting the local receptor density via branched (dendrimeric) structures (cf. FIG. 1b) . For example, a branched structure having a high degree of branching is assigned to a probe with a low melting point and correspondingly, a branched structure having a correspondingly low degree of branching is assigned to a probe with a high melting temperature.

In a first embodiment of the method, one or more predetermined regions are designed with receptors in a different way, i.e. different conditions are chosen for the local receptor concentration from different region sizes, i.e. location sizes for individual receptors, or/and different receptor densities within said regions. According to the invention, those regions which occur frequently in the sample for binding of molecules, for example regions which serve to bind repetitive sequences or regions which serve to bind constitutively highly expressed genes, have an increased local receptor concentration.

Different location sizes may be implemented by way of differently sized synthesis fields during synthesis of the receptors, for example by using an appropriate software. Preferably, the sizes of the individual regions are varied by at least 50%, particularly preferably by at least 100% (based on the size of the smallest region) (see, for example, FIG. 1a).

Different location densities may be implemented via synthetic chemistry using different reagents, for example spacers with different degrees of branching (see, for example, FIG. 1b). The receptor densities of individual regions are preferably varied by at least 50%, particularly preferably by at least 100% (based on the region with the lowest receptor density).

Previous methods do not make possible any large variation possibilities regarding the amounts or local concentrations of the probes, so that it is not possible to carry out an individual adaptation to the greatly varying sample material. The variability described herein of the location area and even of the local receptor concentration per location(location density), which variability may be as large as desired, enables, with the aid of two learning cycles, an adaptation to a defined sample material in order to optimize measurement sensitivity.

Furthermore, individual regions or groups of regions with receptors may have different conditions for receptor-ligand affinity. This is implemented by different receptor lengths or/and different types of receptor building blocks, for example PNA or LNA building blocks, in the individual regions. The receptor length of individual regions is preferably varied by at least 20%, particularly preferably by at least 50% (based on the region having the shortest receptor length).

In a further embodiment, different conditions for the kinetics of receptor-analyte interaction are set in one or more predefined regions with receptors, for example selected from different temperatures or/and temperature profiles in said regions or/and different fluid conditions in said regions.

The temperature may be varied across the entire support, for example across the entire area as a stationary or fluctuating temperature gradient or/and locally across individual regions or groups of regions, for example position-specifically. The control of the temperature over the entire area may be implemented with the aid of a Peltier element or by means of thermally controlled air flow. The temperature may be controlled locally by location-specific irradiation of energy, for example as IR radiation with the aid of a light source matrix, involving illuminating individual locations with an individually set amount of light, resulting in heat production due to absorption. The irradiation here is proportional to the formation of heat and increase in temperature. Alternatively or additionally, the local location area temperature may be regulated by electron flows in conductor tracks which run in the support across individual regions. According to the invention, this temperature control also enables a fluctuating temperature gradient to be set in the individual regions or groups of regions with receptors.

Different double strands which are produced by hybridization of receptors to the supports and target sequences in the sample, which sequences are to be analyzed in a parallel process on a single chip, have different hybridization kinetics and melting curves. The fluctuating temperature gradients described herein and temperatures which can be set locally and individually, for example with the aid of a light source matrix, solve this previously “fundamental” problem of specificity in parallel measurements.

According to the invention, the temperatures in the individual regions are varied preferably by at least 2° C., frequently by at least 5° C. and, in some cases, by at least 10° C.

Another possibility of varying the conditions for the kinetics of receptor-analyte interaction in individual regions of the support is the setting of different fluid movements in one or more different regions of the support. This may involve actively moving the sample during the hybridization process in the fluid processor, for example with the aid of pumps (piston pumps, gas pressure pumps). Preferably the sample is actively moved across the support in a circular flow or/and in a rocking movement.

In the method of the invention, the fluid velocity in individual regions of the support is preferably varied by at least 20%, preferably by at least 50% (based on the region having the lowest fluid velocity).

Active fluid movement enables the sample to be actively moved passed the probe, thereby firstly increasing the rate of hybridization and secondly enabling a separation principle to be utilized in order to separate differently hybridizing sample elements from one another after hybridization (chromatographic principle). In this way it is possible, in combination with fluctuating temperature gradients, to increase specificity and sensitivity. Consequently, according to the invention, the sample may be recycled once or several times across the support under various kinetic conditions. In this context, an increasing temperature profile or/and a decreasing temperature profile or/and a combination of increasing and decreasing temperature profiles may be set per cycle.

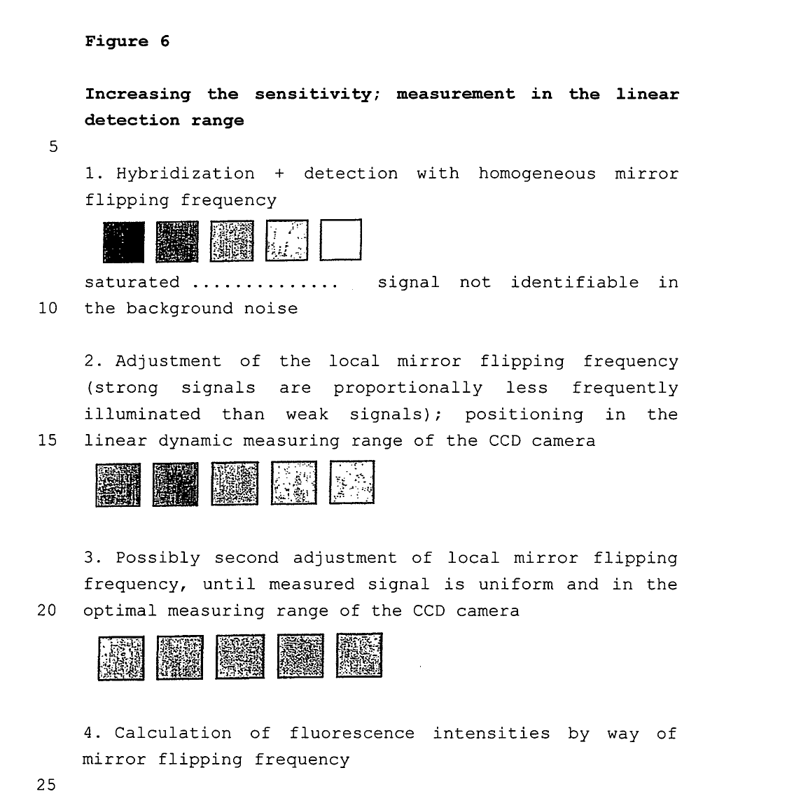

Another parameter which may be varied in the method of the invention is the virtual analyte concentration. To this end, different conditions for determining the analyte concentration are generated. These comprise generating or/and detecting the measured signal in individual regions with different intensity. Preferably, the analyte is detected by way of fluorescence and the different intensity of the measured signal is generated by locally different irradiation with excitation light, preferably via a light source matrix. According to the invention, the individual illumination intensity of the regions varies preferably by at least 50%, particularly preferably by at least 100% (based on the region having the lowest illumination intensity). The locally variable illumination according to the invention via a light source matrix is diagrammatically depicted in FIG. 6.

Previous methods do not enable any individual illumination of individual locations to be controlled to adapt the fluorescence intensities via the amount of excitation light (different illumination of individual locations). After a first test measurement, individual locations may be individually illuminated with the aid of the light source matrix, making it possible to balance different fluorescence emission intensities in individual regions so as not to exceed the linear dynamic measuring range of the detector, for example a CCD camera. This results in an increase especially in the sensitivity and accuracy of quantitative measurements. For example, locations with receptors which have lower melting temperatures are illuminated for a longer time and those with a higher melting point are illuminated for a shorter time so that the signals of the two probes have a comparable intensity. This is particularly important also for applications which do not require quantitative evaluation but are based on a yes/no decision. Examples of these are SNP analyses or resequencing applications in which particular target sequences have a problem, i.e. they are difficult to access for hybridizations, respectively, the corresponding hybridization signals are small and are thus required to be enhanced, and this may then be carried out using local longer illumination times.

According to the method of the invention, the support is preferably a flow cell and/or a microflow cell, i.e. a microfluidic support with channels, preferably with closed channels, in which the predetermined locations with the in each case different immobilized receptors are located. The channels preferably have a diameter in the range from 10 to 10 000 μm, particularly preferably from 50 to 250 μm, and may be designed in principle in any form, for example with a circular, oval, square or rectangular cross section.

The receptors are preferably selected from biopolymers such as, for example, nucleic acids such as DNA and RNA or nucleic acid analogs such as peptide nucleic acids (PNA) and locked nucleic acids (LNA) and also from proteins, peptides and carbohydrates. Particular preference is given to selecting the receptors from nucleic acids and nucleic acid analogs, with binding of the analytes comprising a hybridization.

The method of the invention comprises parallel determination of a plurality of analytes, i.e. a support is provided which contains a plurality of different receptors which may react with in each case different analytes in a single sample. Preference is given to determining by the method of the invention at least 50, preferably at least 100, analytes in the sample in parallel.

The method of the invention is advantageously carried out using an apparatus comprising:
(i) a light source matrix,
(ii) a microfluidic support, having a plurality of predetermined positions at which in each case different receptors selected from nucleic acids and nucleic acid analogs are immobilized on the support,
(iii) a means for delivering fluids to said support and for discharging fluids from said support and
(iv) a detection matrix comprising a plurality of detectors which are assigned to the predetermined regions on the support.

An apparatus of this kind is a light emission detection device disclosed in the German patent applications 198 39 254.0, 199 07 080.6 and 199 40 799.5, which is combined into one apparatus so as to carry out therewith the method of the invention in the form of a cyclic integrated synthesis and analysis. Particular preference is given to using in the apparatus of the invention a programmable light source matrix selected from a light valve matrix, a mirror array and a UV laser array. It is possible to use in the apparatus of the invention two light source matrices, one serving to control the temperature and the other one to detect the measured signals, in the case that the analyte is detected by way of fluorescence. Further preference is given, according to the invention, to using a programmable detection matrix selected from a CCD array, light-sensitive semiconductor structures and electronic detectors. The apparatus of the invention may be utilized for controlled in-situ synthesis of the receptors. Synthesis of the receptors comprises conducting fluid containing receptor synthesis building blocks across the support, location- or/and time-specifically immobilizing said building blocks at the in each case predetermined regions on said support and repeating these steps, until the desired receptors have been synthesized at their in each case predetermined regions. Receptor synthesis furthermore comprises at least one fluid-chemical reaction step or/and at least one illumination step or/and an electrochemical reaction step or/and a combination of such steps.

The present invention will furthermore be illustrated by the following figures:

FIG. 1 diagrammatically depicts the inventive flexible capacity of the chips, which may be useful for increasing the sensitivity and specificity of the hybridization experiments. As FIG. 1a shows, different location sizes are implemented by different synthesis field sizes, with large areas being implemented for depleting repetitive and highly expressed gene sequences and small areas being implemented for the specific probes. FIG. 1b shows how the local receptor density in the individual locations can be increased by spacers branched in a different way.

FIG. 2 shows how it may be possible to increase the specificity of the hybridization process according to the invention by setting a temperature gradient fluctuating with time on the support and by active fluid movement of the sample. The fluctuating temperature profile and the fluid movement (circular flow or/and rocking motion of the fluid) cause detachment of false-positive bonds and, at the same time, concentration of the correct specific bonds.

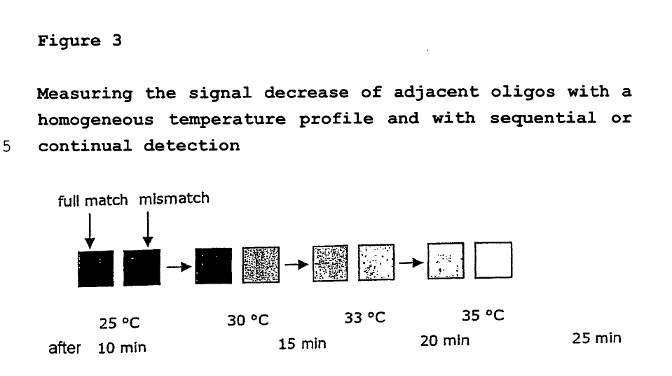

FIG. 3 shows the measurement of the decrease in signal of adjacent receptors having a homogeneous slowly increasing temperature profile, with sequential or continual detection. It is apparent how the temperature increase produces better discrimination between full-match and mismatch regions.

FIG. 4 depicts undesired sequences in the sample being depleted. The hybridization process is carried out in a circular flow with or without fluctuating temperature profile. The conditions of this process are a high local sample concentration, the provision of relatively long receptors for repetitive sequences or/and the setting of a temperature above the melting point of a hybrid between the target sequence and the shorter receptor probes on the support which bind to nonrepetitive sequences of the sample. In the first hybridization cycle, the repetitive sequences hybridize and hybridize more rapidly for kinetic reasons, owing to their higher relative concentration. Thus, the solution is depleted of said repetitive sequences and the depleted solution is stored intermediately in a reservoir. Subsequently, the temperature in the micro-channels may be increased so that the repetitive DNA molecules dehybridize and can then be flushed into another reservoir, for example a waste reservoir. The depleted sample solution is subsequently again hybridized at a lower temperature. Depending on the conditions and sample compositions, the process may also be repeated several times. Alternatively, the depleted sample solution may also be diverted into a “fresh” channel.

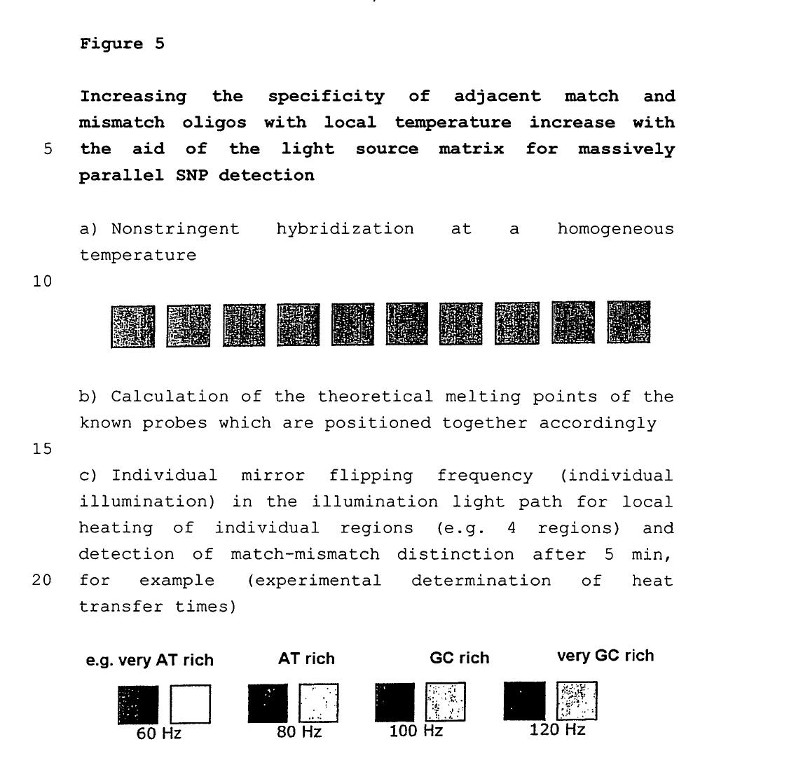

FIG. 5 shows how the local temperature increase increases, with the aid of a light source matrix, the specificity of adjacent match and mismatch receptors and thus makes possible massive parallel SNP detection. A nonstringent hybridization takes place at a homogeneous temperature. Calculating the theoretical melting points of the known probes, it is possible to set in the different regions individual mirror flipping frequencies (individual illumination) in the illumination light path in order to generate in this way local heating of individual regions. This method enables the match-mismatch distinction to be detected. This principle may also be utilized directly in the hybridization. Here, temperature gradients arise which make possible simultaneous hybridization of a multiplicity of DNA strands having different melting temperatures.

FIG. 6 diagrammatically shows how detection with a homogeneous mirror flipping frequency is carried out after a hybridization process. Saturated regions are recognizable as are, however, also regions in which the signal is lost in the background noise and thus cannot be identified. In order to obtain increased sensitivity, the illumination with excitation light must be adjusted, for example via the local mirror flipping frequencies, with strong signals being proportionally less frequently illuminated than weak signals. A positioning takes place in the linear dynamic measuring range of the detector, for example a CCD camera. It may be necessary to carry out a second adjustment of the local mirror flipping frequency, until the measured signal is uniform and until the signal is in the optimal measuring range of the detector. The fluorescence intensity is then calculated via the mirror flipping frequency.

### EXEMPLARY EMBODIMENTS

### EXAMPLE 1

### Expression Profiling with a Complete Yeast Genome (6000 Genes)

Supports are prepared, containing in each case 500 locations for GAPDH, actin, and other genes known to be highly expressed and having in each case one location for all other, rarely expressed genes. A hybridization experiment is carried out. According to the measured signal intensities, the individual locations and the individual location densities are adjusted (equilibration of melting temperatures). The hybridization process is repeated, prolonging the detection times in order to increase the sensitivity in the linear measuring range. The redundant locations are integrated to give one measured value.

### EXAMPLE 2

### Sequencing by Means of Hybridization (SbH) with a Human BAC Sequence

Half of a support is charged with a multifunctional spacer and with receptor mixtures which are complementary to the repetitive regions and to the vector sequence. These receptors have a length of up to 50 bases. The other half of the support is charged with shorter receptors in order to resequence regions from target genes. A temperature gradient is applied, with elevated temperatures being set for repetitive regions, i.e. for long receptors, in order for these regions not to be depleted of specific sequences due to false hybridization, and low temperatures being set for specific regions, i.e. short receptors. The BAC DNA is randomly fragmented and the hybridization process is carried out subsequently. The hybridization may be carried out cyclically in order to make use of the effect of reassociation kinetics. The signals are detected in the specific range with an improved signal-to-background ratio.

### EXAMPLE 3

### EXAMPLE 3a

### Sequencing by Means of Hybridization (SbH) with Increased Specificity via a Fluctuating Temperature Gradient

This example is diagrammatically depicted in FIG. 2. A fluctuating temperature gradient is applied. During this time, a hybridization is carried out in a circular flow. With the aid of fluid convection in combination with the fluctuating thermal control, a thermodynamic equilibrium is set by way of detaching false-positive hybridization events (kinetically controlled local thermodynamic minima).

### EXAMPLE 3b

### Sequencing by means of Hybridization (SbH) with Increased Specificity via Online Observation after Hybridization and Temperature Increase

This example is depicted diagrammatically in FIG. 3. Match and mismatch receptors are positioned directly adjacent to each other. A hybridization with a target nucleic acid is carried out in a fluctuating temperature gradient. The hybridization temperature is slowly increased, while the hybridization signal is measured. Detection is carried out by way of online or interval observation using an online CCD camera.

### EXAMPLE 3c

### Sequencing by means of Hybridization (SbH) with Increased Specificity via Local Temperature Control Prior to or/and after Hybridization

This example is depicted diagrammatically in FIG. 5. Match and mismatch receptors are positioned directly adjacent to each other on the support. The hybridization is carried out with a target nucleic acid in a fluctuating temperature gradient. The hybridization temperature is set and locally different heat quantities are then introduced with the aid of local illumination by means of a light source matrix. To this end, preference is given to utilizing IR rays as light source. Determination of a difference in the intensity of individual receptor pairs at a desired time results in a single base match-mismatch distinction.

1. A method of determining analytes, comprising the steps
(a) providing a support having a plurality of predetermined regions at which in each case different receptors are immobilized on said support,
(b) contacting said support with an analyte-containing sample and
(c) determining the analytes via their binding to the receptors immobilized on the support,
characterized in that
for predetermined regions or groups of regions with receptors in each case different conditions (i) for local receptor concentration, (ii) for receptor-ligand affinity, (iii) for kinetics of receptor-analyte interaction or/and (iv) for virtual analyte concentration are provided.

2. The method as claimed in claim 1, characterized in that the different conditions for local receptor concentration are selected form different region sizes or/and different receptor densities within the regions.

3. The method as claimed in claim 2, characterized in that the regions have an increased local receptor concentration to bind molecules which frequently occur in the sample.

4. The method as claimed in claim 2, characterized in that the regions have an increased local receptor concentration to bind repetitive sequences.

5. The method as claimed in claim 2, characterized in that the regions have an increased local receptor concentration to bind constitutively highly expressed genes.

6. The method as claimed in claim 2, characterized in that the sizes of individual regions are varied by at least 50%, preferably by at least 100%.

7. The method as claimed in claim 2, characterized in that the different receptor densities of individual regions are implemented using spacers with different degrees of branching.

8. The method as claimed in claim 2, characterized in that the receptor densities of individual regions are varied by at least 50%, preferably by at least 100%.

9. The method as claimed in claim 1, characterized in that the different conditions for receptor-ligand affinity are implemented by different receptor lengths within the regions or/and different types of receptor building blocks.

10. The method as claimed in claim 9, characterized in that the receptor lengths of individual regions are varied by at least 20%, preferably by at least 50%.

11. The method as claimed in claim 1, characterized in that the different conditions for the kinetics of receptor-analyte interaction are selected from different temperatures or/and temperature profiles in the regions or/and different fluid conditions in said regions.

12. The method as claimed in claim 11, characterized in that the different temperature control in the regions is generated by local energy irradiation, preferably via an IR light source matrix.

13. The method as claimed in claim 11, characterized in that a fluctuating temperature gradient is set in individual regions or groups of regions with receptors.

14. The method as claimed in claim 11, characterized in that the temperatures of individual regions are varied by at least 2° C., preferably by 5° C., preferably by at least 10° C.

15. The method as claimed in claim 11, characterized in that the sample is actively moved across the support in a circular flow or/and in a rocking motion.

16. The method as claimed in claim 11, characterized in that the fluid velocities in individual regions are varied by at least 20%, preferably by at least 50%.

17. The method as claimed in claim 11, characterized in that a sample is recycled across the support once or several times under various kinetic conditions.

18. The method as claimed in claim 11, characterized in that an increasing temperature profile or/and a decreasing temperature profile or/and a combination of increasing and decreasing temperature profiles per cycle is set.

19. The method as claimed in claim 1, characterized in that the different conditions for the virtual analyte concentration comprise generating or/and detecting the measured signal in individual regions with different intensity.

20. The method as claimed in claim 19, characterized in that the analyte is detected by way of fluorescence and the different intensity of the measured signal is generated by locally different irradiation of excitation light, preferably via a light source matrix.

21. The method as claimed in claim 19, characterized in that the illumination intensities in individual regions vary by at least 50%, preferably by at lest 100%.

22. The method as claimed in claim 1, characterized in that a microfluidic support with channels, preferably closed channels, in which the predetermined regions with immobilized receptors are located, is used.

23. The method as claimed in claim 1, characterized in that the receptors are selected from biopolymers such as, for example, nucleic acids, nucleic acid analogs, proteins, peptides and carbohydrates.

24. The method as claimed in claim 23, characterized in that the receptors are selected from nucleic acids and nucleic acid analogs and that binding of the analytes to the receptors encompasses a hybridization.

25. The method as claimed in claim 1, characterized in that a plurality of analytes, preferably at least 50 analytes, and particularly preferably at least 100 analytes, are determined in parallel in the sample.

26. The method as claimed in claim 1, characterized in that the analytes are determined using an apparatus, comprising
(i) a light source matrix,
(ii) a microfluidic support,
(iii) a means for delivering fluid to said support and for discharging fluids from said support and
(iv) a detection matrix.

27. The method as claimed in claim 26, characterized in that a programmable light source matrix selected from a light valve matrix, a mirror array and a UV laser array is used.

28. The method as claimed in claim 27, characterized in that a programmable detection matrix selected from a CCD array, light-sensitive semiconductor structures and electronic detectors is used.

29. The method as claimed in claim 1, characterized in that the receptors are synthesized in situ on the support.

30. The method as claimed in claim 29, characterized in that synthesis of the receptors comprises: conducting fluid having receptor synthesis building blocks across the support, location-or/and time-specifically immobilizing said building blocks at the in each case predetermined regions on said support and repeating these steps until the desired receptors have been synthesized at the in each case predetermined regions.

31. The method as claimed in claim 29, characterized in that synthesis of the receptors comprises fluid-chemical reaction steps or/and illumination steps or/and electrochemical reaction steps.

32. An apparatus for determining an analyte, comprising a support having a plurality of predetermined regions at which in each case different receptors are immobilized on said support, characterized in that said predetermined regions with receptors have, at least partially, a different local receptor concentration.

33. An apparatus for determining an analyte, comprising a support having a plurality of predetermined regions at which in each case different receptors are immobilized on said support, characterized in that means are provided in order to vary the kinetics of the receptor-analyte interaction in the predetermined regions.

34. An apparatus for determining an analyte, comprising a support having a plurality of predetermined regions at which in each case different receptors are immobilized on said support, characterized in that means are provided in order to vary the virtual analyte concentration in the predetermined regions.
